(19) United States
(12) Patent Application Publication
(10) Pub. No.: US 2007/0179815 A1
(43) Pub. Date: Aug. 2, 2007

(54) MEDICAL SCREENING SYSTEM AND METHOD

(75) Inventors: David T. Vining, Andover, MA (US); Bert R. Vining, Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: HeartScreen America, Inc., Wilmington, DE (US)

(21) Appl. No.: 11/624,813

(22) Filed: Jan. 19, 2007

Related U.S. Application Data
(60) Provisional application No. 60/760,501, filed on Jan. 20, 2006.

(51) Int. Cl. G06F 19/00; A61B 5/00; A61B 5/04

(52) U.S. Cl. 705/3; 600/509; 600/300

(57) ABSTRACT

Systems and methods for screening large client populations (e.g., students) to identify those clients susceptible to a medical condition (e.g., a cardiac condition) are provided. In general, the system allows for a large number of clients to be tested at a convenient location (e.g., a school), on-site identification of those clients at immediate risk, and over-read of each test result (both normal and abnormal test results) at a later date by a physician capable of gaining access to the records via a secure computer connection. The system further includes various servers and network connections capable of creating and maintaining a secure central database, creating and updating electronic client medical records, down-loading data (e.g., the electronic records) from the central database to portable computing units, collecting client testing data, up-loading and integrating the testing data into the secure central database, and providing secure access to authorized end-users.

### RELATED APPLICATIONS

This application claims priority to U.S. provisional patent application Ser. No. 60/760,501, filed on Jan. 20, 2006, and entitled “Medical Screening Method and System,” the provisional application being incorporated herein by reference in its entirety.

### FEDERALLY SPONSORED RESEARCH

Not applicable.

### FIELD OF INVENTION

This invention relates to methods and systems for efficiently screening a large number of clients for a medical condition. More particularly, the invention relates to a screening method and system for screening large client populations for indications of a cardiac condition(s).

### BACKGROUND OF THE INVENTION

While a handful of dedicated foundations are working to get the word out, people have not yet come to understand the pervasiveness of life threatening heart defects. Sudden cardiac death is really an unrecognized epidemic. In fact, according to some reports, sudden cardiac death is the most common cause of death. One out of every two heart deaths is due to sudden cardiac death, and in general, one in every five deaths is related to sudden cardiac death. Anything that could even marginally reduce sudden death risk could have a dramatic impact.

Some reported facts relating to sudden cardiac death:

Each month, 250 to 600 young Americans die suddenly and unexpectedly from cardiac arrhythmia syndromes.

One person in every 350 has either hypertrophic cardiomyopathy (HCM) or one of the other causes of cardiac arrhythmia syndrome.

One person in every 500 has HCM, an excessive thickening of the heart muscle that can lead to heart rhythm disturbances and sudden death.

HCM is the leading cause of Sudden Unexpected Death in those under age 35.

HCM is more common in the U.S. than childhood leukemia, cystic fibrosis, Parkinson's disease, and multiple sclerosis.

HCM is a genetic condition, meaning affected genes are typically passed from one generation to the next. Once an individual is identified as having HCM, other family members should be screened as well. Screening hearts can save lives.

Hypertrophic Cardiomyopathy and the other leading causes of SUD, such as Arrhythmogenic Right Ventricular Dysplasia, Myocarditis, Dilated Cardiomyopathy, Q-T Segment Prolongation, and Primary Conduction Abnormalities are detectable and together account for more than 60% of all instances of Sudden Unexpected Death.

Most heart defects cannot be detected by the traditional physician's physical examination but a non-invasive heart screening can identify those at risk of HCM and other leading causes in up to 97% of cases.

The unexpected death of an apparently healthy person is a tragedy, causing great anguish in the community as well as the family. These tragedies continue to occur today because patients are not screened routinely for the underlying heart conditions. ECG screening is not a routine part of the traditional physician's physical examination. As a result of the number of athletes claimed by sudden cardiac death, a number of national and international sports governing bodies have adopted the Lausanne Recommendations, which calls for pre-participation cardiovascular screening of all athletes. According to the Recommendations, screening should include four elements: the personal cardiac history of the athlete, his or her family cardiac history, an electrocardiogram (ECG) and a physical examination (by a Primary Care Physician). Young athletes suffer Sudden Unexpected Death at a rate thought to be two to three times greater than their less-active peers because exercise—while not the cause of cardiac defects—can push an already compromised heart over the edge. Families who now know the perils of Hypertrophic Cardiomyopathy (the leading cause of SUD in athletes) because they have suffered the loss of a loved one, often say that their doctors never suggested that their children undergo a heart screen.

### SUMMARY OF THE INVENTION

In one aspect, the present invention provides a method for screening a population for a medical condition. In an exemplary embodiment, the method includes screening/testing a group of students at a location (e.g., a school) for a cardiac condition. In general, the method can include (A) establishing a testing site at a location remote from a healthcare facility, the testing operation including at least one healthcare clinician and at least one mobile computing unit; (B) obtaining or creating an electronic medical record for a client from among the population and storing that electronic medical record on the at least one mobile computing unit; (C) testing of the client for the condition by the healthcare clinician using a digital medical device connected to the mobile computing device; (D) adding data from the testing to the client's electronic medical record; (E) uploading the client's electronic medical record from the mobile computing device to a Data Management Server associated with the healthcare facility; and (F) providing authorized end-users with access to the client's electronic medical record. The method can also include repeating steps (A) through (F) in order to test the entire population. Additionally, the mobile computing unit can be in communication with a storage device, the storage device capable of maintaining client records while at the remote testing location.

In one embodiment, the method can include a plurality of healthcare clinicians, each having a testing computing device in communication with the mobile computing device, the testing devices being coupled to digital medical devices. Further, each clinician can have two digital medical devices so that one client from the population can be prepared for testing while another client from the population is being tested. In addition to an On-Site Team at the testing location, the system can also include an on-call medical professional (i.e., a physician), wherein the electronic medical record, including testing data, can be forwarded electronically to the on-call medical professional at a remote location at least for clients whose testing indicates a likelihood that the medical condition is present.

In another aspect, the method can include providing a Screening Management System (“SMS”) portal which can allow a client to establish an electronic client medical record by providing various types of information via an internet connection, with the electronic medical record stored in a secure Data Management Server (“DMS”). Next, the method can include down-loading the electronic medical records to a mobile computer unit, the mobile computer unit capable of being moved to a remote testing facility. Further, the method can include coupling a digital medical device (e.g., an ECG device) to the mobile computer unit, the digital medical device capable of performing a client test and incorporating test data to the electronic client record to produce an updated electronic record. Following testing, the method can include up-loading the electronic client records from the mobile computer unit to the data management server, and providing an off-site medical professional (i.e., physician) with access to the electronic client record via a terminal server, the terminal server being coupled to the data management server. Additionally, the method can include providing the client with access to the client's electronic medical record via the internet connection.

In a further aspect, the present invention can provide a system for screening a population of clients for a medical condition. The system can include a data management server located at a healthcare facility, the data management server in communication with an SMS portal, the SMS portal assessable to a client via an internet connection. Additionally, the data management server can be capable of creating an electronic client record containing various information provided by the client via the SMS portal. Further, the system can include at least one mobile computer unit capable of moving from the healthcare facility to the remote testing location, the mobile computer unit further capable of receiving electronic records downloaded from the data management server. The mobile computer unit can be coupled to a digital medical device capable of performing a client test and directly incorporating test results into the client's electronic record. The mobile computer unit can be further capable of uploading the electronic medical record to the data management server. Further, the system can include a terminal server capable of allowing an off-site medical professional access to the electronic medical record. The system can also include a plurality of testing computer units in communication with the mobile computer unit.

### BRIEF DESCRIPTION OF THE DRAWINGS

FIG. 1 is a block diagram of a general overview of an exemplary embodiment of the presently disclosed invention;

FIG. 2 is a block diagram of stages of the presently disclosed method;

FIG. 3 is a block diagram of steps included in an embodiment of an ‘Initiation Stage’ of the presently disclosed method;

FIG. 4 is a block diagram of steps included in an embodiment of a ‘Testing Stage’ of the presently disclosed method;

FIG. 5 is a representation of an exemplary embodiment of a testing location;

FIG. 6 is a block diagram of various tests which can be performed during the Testing Stage;

FIG. 7 is a block diagram of steps included in an embodiment of a ‘Results Analysis and Reporting Stage’;

FIG. 8 is a block diagram of steps included in an embodiment of analyzing a client's pulse rate;

FIG. 9 is a block diagram of steps included in an embodiment of analyzing a client's blood pressure; and

FIG. 10 is a block diagram of steps included in an embodiment for reviewing test results by an off-site medical professional as well as reporting results to clients.

### DETAILED DESCRIPTION

The presently disclosed invention provides systems and methods for the efficient screening of a large client population (e.g., students/student athletes) in order to identify those members of the population susceptible to a medical condition (e.g., a cardiac condition). In general, the system allows for a large number of clients to be tested at a convenient location (e.g., a school), on-site identification of those clients at immediate risk, and over-read of each test result (both normal and abnormal test results) at a later date (within about a week, for example) by a medical professional, the medical professional capable of gaining access to the relevant electronic records via a secure computer connection (thereby optimizing the medical professional's time). The system further includes various servers and network connections capable of creating and maintaining a secure central database, creating and updating electronic client medical records, down-loading data (e.g., the electronic records) from the central database to portable computing/testing units, collecting client data via digital medical devices coupled to the portable computing units, up-loading and integrating the testing data into the secure central database, and providing authorized end-users (e.g., the client themselves or an off-site medical professional) secure access to the central database. As will be discussed in detail below, the presently disclosed systems and methods provide powerful tools capable of efficient, preventative client screening.

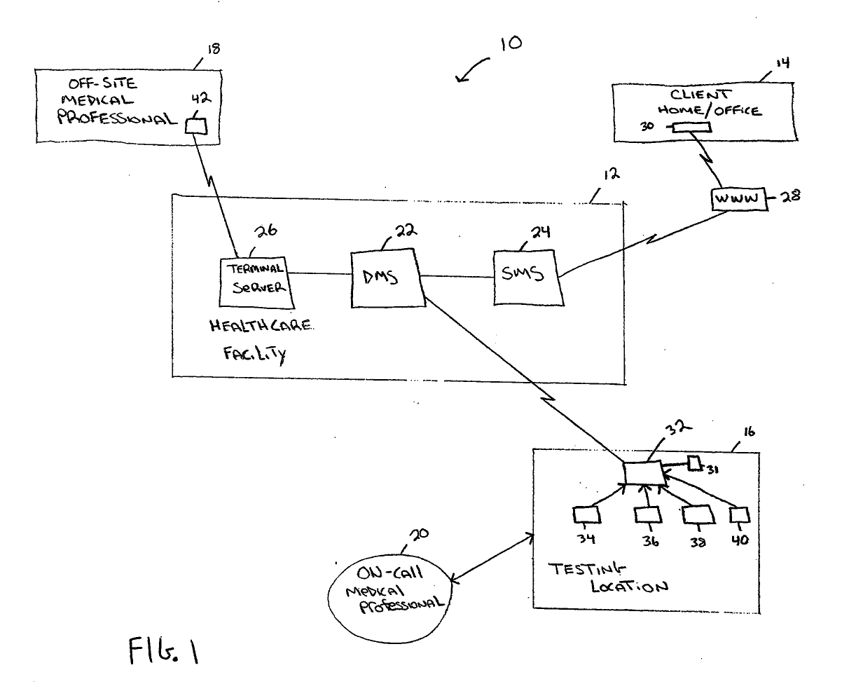

As shown in FIG. 1, a system 10 and method according to one embodiment of the invention can include various inter-connected computers located and portable between various locations. In general, the locations can include a healthcare facility 12, a client located at an off-site location (e.g., home or office) 14, a testing location 16, an on-call medical professional 20 (accessible via phone, e-mail or present at the testing location 16), and an off-site medical professional 18. As shown, a secure data management server (“DMS”) 22 is located at the healthcare facility 12. The DMS 22 can be capable of managing and securely storing (i.e., accessible only by authorized persons) all client records, creating and maintaining electronic client records, producing reporting letters, scheduling testing sessions and locations, etc. As such, the DMS 22 can be considered to be the central-hub of the system 10. As will be discussed in detail below, various end-users (e.g., off-site medical professionals and/or clients) can be provided with secure access to select data stored in the DMS 22. Those skilled in the art will appreciate that various types of data management servers 22 are within the spirit and scope of the present invention. In an exemplary embodiment, the DMS 22 is an IQmark® DMS (available from Midmark Diagnostics Group of Versailles, Ohio).

Looking at FIG. 1, the DMS 22 is in communication with a Screening Management System (“SMS”) portal 24 capable of allowing clients electronic access to the system 10 in order to schedule a test, input client information (e.g., client medical history, family medical history, etc.) in order to create an initial electronic client record and/or review client's own electronic record following testing, the SMS portal 24 being any combination of hardware and/or software capable allowing such access. More specifically, the client can access the SMS portal 24 through an internet connection 28 from a client's personal computer 30 located at client's home/office 14. These steps will be discussed in detail below.

Where testing has been scheduled for a population of clients (e.g., all students at a school), the electronic records (one for each client) can be down-loaded from the DMS 22 to a mobile computing unit 32 for deployment at the testing location 16. In order to protect the secrecy of client records, only the information necessary for testing (e.g., client name, time of testing, etc.) can be downloaded from the DMS 22 to the mobile computing unit 32. The mobile computing unit 32 can further include a storage device 31 capable of maintaining all electronic records while at the remote location 16. Further, the mobile computing device 32 can be coupled to digital medical devices (e.g., digital electrocardiographs) in order to obtain test results and incorporate the results directly into the associated electronic record. In one embodiment, the mobile computer unit 32 can be in communication with a plurality of testing computer units 34, 36, 38, 40 each of which also coupled to digital medical devices and in communication with the storage device 31 so as to allow for multiple, simultaneous screenings. The mobile computing unit 32 can be any computer unit capable of being moved from the healthcare facility 12 to the testing location 16, the mobile unit 32 further capable of coupling to a digital medical device (e.g., a digital ECG medical device) capable of obtaining test data and incorporating the data into the electronic record 114. For example, the mobile computer unit 32 can be a laptop, a personal data assistant (“PDA”), a portable desk top computer, or any other type of portable computing unit. In an exemplary embodiment, the mobile computing unit 32 is a lap top computer. Like the mobile computer unit 32, the testing computer units 34, 36, 38, 40 can be any type of portable computer unit capable of moving from the healthcare facility 12 to the remote testing location 16 and further being capable of coupling to the digital medical device.

To deploy the various portable computing units 32, 34, 36, 38, 40, a Team (e.g., a Team Leader and a group of clinicians) can be dispatched with the units 32, 34, 36, 38, 40 to the remote testing center 16. As further indicated in FIG. 1, during testing, an on-call medical professional 20 (e.g., a cardiologist) can be in communication (by e-mail, phone, or present on location) with the Team so as to consult with Team Members regarding abnormal test results. As will be discussed below, following testing (or periodically throughout the day), the data can be up-loaded from the storage device 31 to the secure DMS 22. Following testing, all client data can be cleared from any mobile unit 32, 34, 36, 38, 40 in order to further ensure secrecy and security of client data.

An off-site medical professional 18 (i.e., a physician) can electronically access the specific client records via the medical professional's computer 42 by contacting a terminal server 26 (e.g., a Microsoft terminal server) in communication with the DMS 22. Such access can be provided so as to allow the physician to review (i.e., over-read) all test results. After this final review, the off-site medical professional can confirm or dispute the initial results and provide the healthcare facility 12 with recommendations, if any, on how to proceed (e.g., inform client to seek medical attention).

After all relevant information is taken into account, the DMS 22 can create a reporting letter (containing qualitative and/or quantitative information) to be sent to the client for their records. Additionally, the system 10 can allow for the client to access their electronic client record via the SMS portal 24.

As shown, the system 10 can allow for an immediate response to an emergency situation by the On-Site Team and the on-call medical professional while allowing for the off-site physician to review (i.e., over read) all test results (both normal and abnormal) at a later time (for example within about 5 days). System 10 can thereby providing an efficient screening procedure. The following discussion provides greater detail of the general overview provided above.

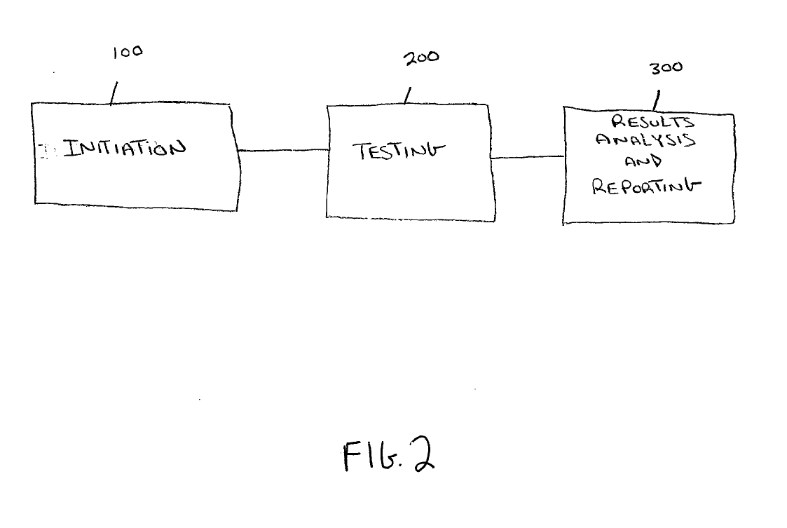

As shown in FIG. 2, a method according to one embodiment of the invention can include three stages: the ‘Initiation Stage’ 100, the ‘Testing Stage’ 200, and the ‘Results Analysis and Reporting Stage’ 300. These stages are presented for the sole purpose of facilitating the following discussion. Those skilled in the art will readily appreciate that various steps can be performed in multiple stages or in different stages. Furthermore, various alternative embodiments can include the addition or subtraction of any number of steps to any stage of the method. All of these alternatives are within the spirit and scope of the present invention.

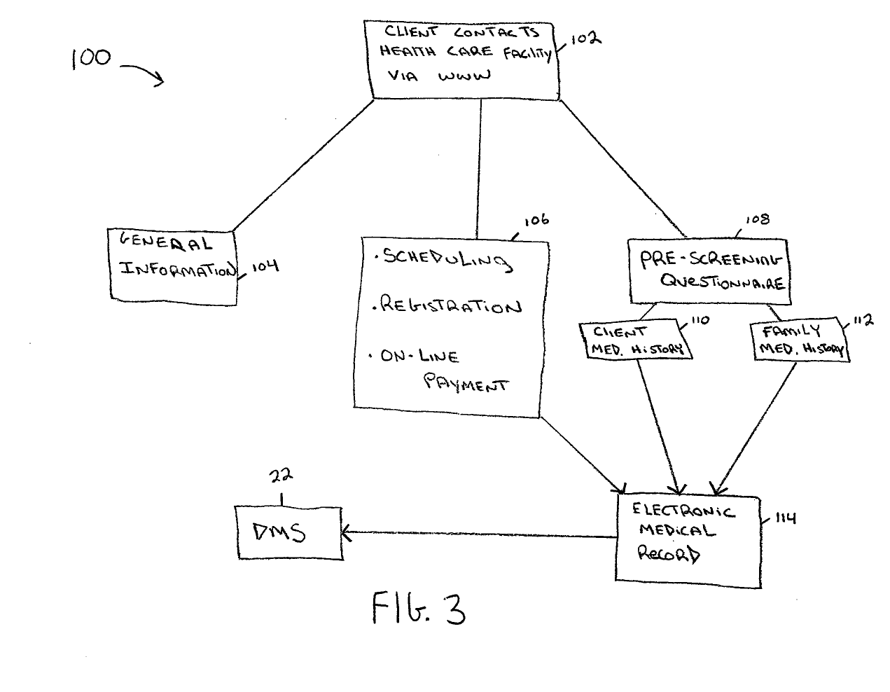

The Initiation Stage 100 generally includes a client contacting the healthcare facility 12 (via internet/telephone/walk-in) in order to schedule a testing and provide information in order to create an initial electronic record. In the exemplary embodiment of FIG. 3, a client contacts the healthcare facility 12 via an internet connection (Step 102). The web page can include various information regarding specific medical conditions (e.g., cardiac conditions) for the client's education (Step 104). Next, the website can allow the client to schedule a testing, register, and/or provide payment for the testing (Step 106). After a test has been scheduled, the client can be asked to fill-out a pre-screening questionnaire (Step 108). The questionnaire can ask for any type of information related to the testing. For example, the questionnaire can ask for the client's medical history (Step 110) and/or the client's family medical history (Step 112). All information can be used to create a client-specific electronic medical record 114. In an alternative embodiment, the client can walk-in to the testing location 16 and provide all such information in order to create an electronic client record and schedule testing.

Regarding the electronic record 114, the DMS 22 can utilize various computerized client record system software and an appropriate database infrastructure. One such computerized client record system that can be applied is “VISTA,” available from the Department of Veterans Affairs in Tuscaloosa, Ala. This automated information system has been developed by the Department of Veterans Affairs (VA) in its medical facilities and it includes extensive clinical and administrative capabilities. The original Veterans Health Information Systems and Technology Architecture (VISTA), supporting ambulatory and inpatient care, added to the original system with the release of the Computerized Patient Record System (CPRS) for clinicians in 1997. CPRS provides a single interface for health care providers to review and update a patient's medical record and the ability to place orders including medications, special procedures, x-rays, patient care nursing orders, diets, and laboratory tests. CPRS is flexible enough to be implemented in a wide variety of settings for a broad spectrum of health care workers, and provides a consistent, event-driven, windows-style interface. VISTA Imaging can also be deployed. VISTA Imaging provides a multi-media, online patient record that integrates traditional medical chart information with medical images including x-rays, pathology slides, video views, scanned documents, cardiology exam results, wound photos, dental images, endoscopies, etc. A person of ordinary skill in the art will recognize that other patient record systems could be used, or that an entirely new system could be designed for use with the presently disclosed system and method.

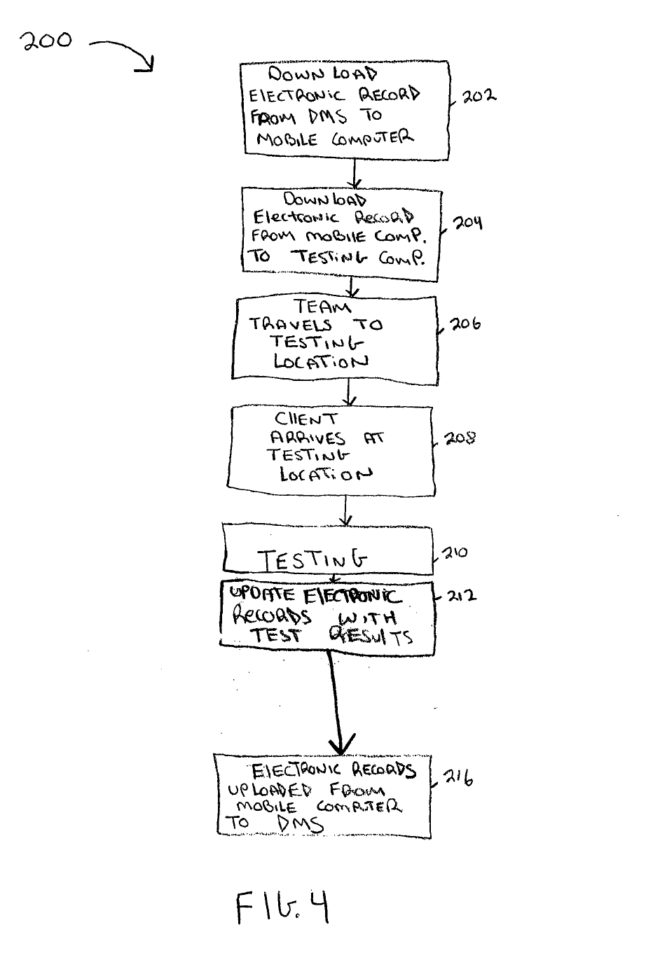

Following the Initiation Stage 100, the system 10 can proceed to a Testing Stage 200. FIG. 4 is an overview of an exemplary embodiment of the Testing Stage 200. As shown, in anticipation of testing, the relevant electronic records 114 (i.e., the records corresponding to those clients to be tested at the testing location 16) can be down-loaded from the DMS 22 to the mobile computing unit 32 (step 202), the mobile unit 32 in communication with the storage device 31 capable of maintaining electronic records 114 at the remote location 16. In order to protect client information, the down-loaded information can include only information necessary for testing and the remaining information can be maintained in the secure DMS 22. Further, the mobile computer unit 32 can be in communication with a plurality of testing computer units 34, 36, 38, 40 (Step 204), the testing units 34, 36, 38, 40 also capable of coupling to digital medical devices in order to allow for multiple test to be performed simultaneously.

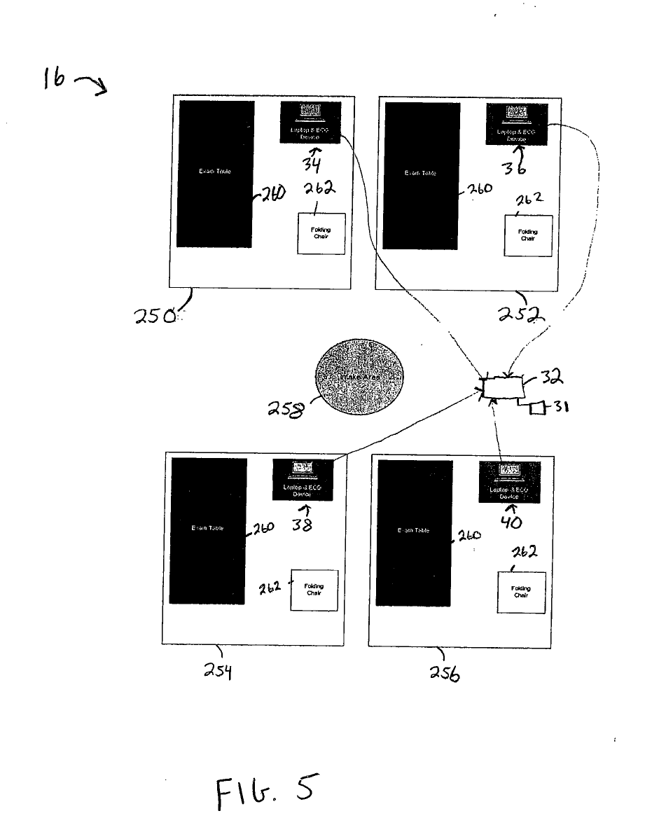

Once the appropriate records are downloaded from the DMS 22 to the mobile computer unit 32, a Team can travel to the remote testing location 16 (Step 206). The Team can include any number of members as are deemed necessary by the healthcare facility 12. Typically, the number of Team Members will be dependent on the number of clients to be tested on a specific day. In an exemplary embodiment, the Team can include: an intake member, the intake member responsible for greeting and directing clients; a Team Leader; and, a plurality of clinicians responsible for performing the actual testing. Regarding the testing location 16, the location 16 can be any location 16 capable of housing a large number of clients as well as being convenient for the clients. The location 16 can be, but does not need to be, a medical facility. In an exemplary embodiment, the testing location 16 is a school and the clients are students. FIG. 5 provides a general overview of an embodiment of a testing location 16 wherein the location 16 includes an intake area 258 and a plurality of testing areas 250, 252, 254, 256. As shown, each area 250, 252, 254, 256 includes an exam table 260, a chair 262, and a testing computer 34, 36, 38, 40 coupled to a digital medical device. In one embodiment, each testing area can include multiple digital medical devices so that while a first client is being tested with a first device, a second client can be prepared with the second device.

Returning to FIG. 4, in the next step of the outlined embodiment, the clients arrive at the testing location 16 (Step 208) at their scheduled testing time. Upon arrival, a client may check-in at the client-intake area 258 wherein the intake member can greet the client and direct the client to a designated testing area 250, 252, 254, 256. Next, a clinician(s) assigned to the testing area 250, 252, 254, 256 can perform any number and/or type of tests, the test results being captured by the testing computer 34, 36, 38, 40 and incorporated into the client's electronic record 114 (Step 212). The types of tests and analysis of results will be discussed in detail below. At the end of the day (or periodically during the day), the electronic records 114 can be uploaded from the storage device 31 to the DMS 22 located at the healthcare facility 12 (Step 216).

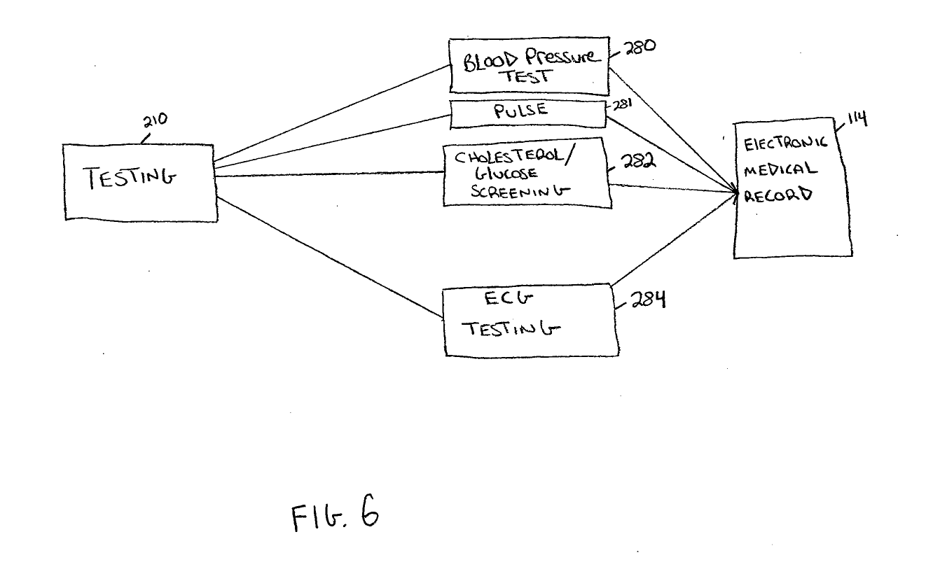

As stated above, the presently disclosed system and method can include any type of testing deemed necessary and/or desirable in light of the purpose of the screening. In a particularly preferred embodiment, the screening is performed in order to identify a cardiac condition (e.g. hypertrophic cardiomyopathy or any other cause of cardiac arrhythmia syndrome). FIG. 6 provides an embodiment of tests typically included when screening for such a condition. As shown, testing (Step 210) can include a blood pressure test (Step 280), a pulse measurement (Step 281), a cholesterol/glucose test (Step 282) and/or ECG testing (Step 284). As will be apparent to those skilled in the art, any of these tests can be removed, performed in any order, performed multiple times (in order to verify results), or additional tests can be added. The data resulting from each test can be incorporated (either manually or automatically) into a client's electronic medical record 114.

An electrocardiogram (“ECG”) is a graphic produced by an electrocardiograph, which records the electrical activity of the heart over time. It is the standard of cardiac rhythm analysis and plays a key role in the screening and diagnosis of cardiovascular disease. In performing ECG testing (Step 284), any testing device capable of producing an ECG and coupling to the testing computer 34, 36, 38, 40 such that the results are directly incorporated into a client's electronic record are within the spirit and scope of the present invention. In an exemplary embodiment, the ECG testing device is an IQmark® digital ECG (available from Midmark Corp. of Versailles, Ohio.)

Regarding cholesterol/glucose testing (Step 282), any device capable of accurately determining a client's cholesterol and/or glucose level is within the spirit and scope of the present invention. In an exemplary embodiment, cholesterol and/or glucose levels are tested with a Cholestech LDX® Analyzer (available from Cholestech of Hayward, Calif.) wherein results can be manually entered into the testing computer 34, 36, 38, 40 and incorporated into the client's electronic record 114. Finally, blood pressure and/or pulse can be checked via a stethoscope and a sphygmomanometer. Like cholesterol results, these results can be manually entered into the testing computer and incorporated into a client's electronic record 114.

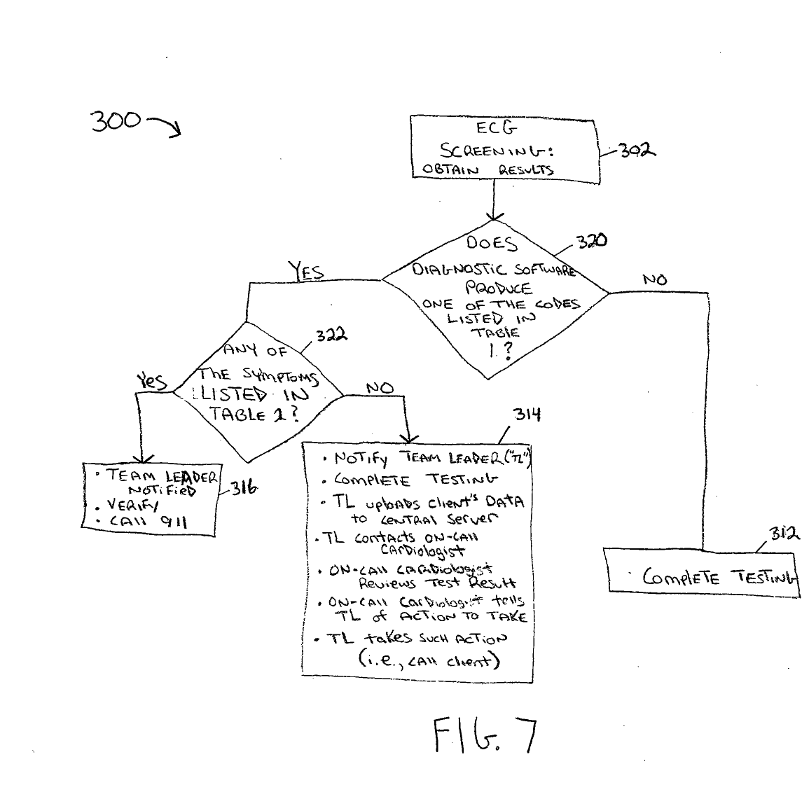

As test results are generated, the On-Site Team (e.g., Team Leader and clinicians) can review test data and identify abnormal test results. If results are identified indicating an emergency situation, the Team can contact an on-call medical professional (e.g., by e-mail, phone, video-conferencing, or in person) to provide recommendations (such as advising client to seek immediate medical treatment). The block diagrams of FIGS. 7-10 provide examples of steps the On-Site Team can take in reviewing test date, categorizing clients by test results, and handling emergency situations.

FIG. 7 is a representation of steps which can be included in a ‘Results Analysis and Reporting Stage’ 300. As shown, following ECG testing and production of test results (step 302), the clinician can determine whether the diagnostic software produces one of the codes listed in Table 1 below (Step 320):

TABLE 1

| CODE | MEANING |
|---|---|
| 2AVB | Second degree AV block |
| 3AVB | Third degree AV block |
| AFIB | Atrial fibrillation |
| AFLT | Atrial flutter |
| ALMI | Anterolateral myocardial infarction |
| AMI | Anterior myocardial infarction |
| APMI | Apical myocardial infarction |
| ASMI | Anteroseptal myocardial infarction |
| BRU | Brugada Syndrome |
| HLMI | High-lateral myocardial infarction |
| ILMI | Inferolateral myocardial infarction |
| IMI | Inferior myocardial infarction |
| IPLMI | Inferoposterolateral myocardial infarction |
| IPMI | Inferoposterior myocardial infarction |
| JRHYT | Junction rhythm |
| LMI | Lateral myocardial infarction |
| LNGQT | Long QT interval |
| MI | Myocardial infarction |
| MYOIN | ST-T suggests myocardial infarction (no location |
| | specified) |
| PMI | Posterior myocardial infarction |
| VTACH | Ventricular tachycardia |
| WPW | Wolf-Parkinson-White syndrome |
| WQTAC | Wide QRS tachycardia |

If none of these codes are produced, the clinician can proceed to complete testing (Step 312). If one or more of these codes are produced, the clinician can determine whether client is exhibiting any of the symptoms listed in Table 2 below (Step 322):

TABLE 2
SYMPTOM

Chest Pain
Shortness of Breath
Nausea/Vomiting
Dizziness, Weakness or Fainting
Profuse Sweating
Low Blood Pressure

If any of these symptoms are present, the client can be considered as an emergency situation wherein the clinician, Team Leader, and on-call medical professional can review results and recommend the client to emergency assistance (Step 316). If none of these symptoms are present, clinician can inform Team Leader of situation, Team Leader can contact on-call medical professional who can review results and provide recommendations, if any (Step 314).

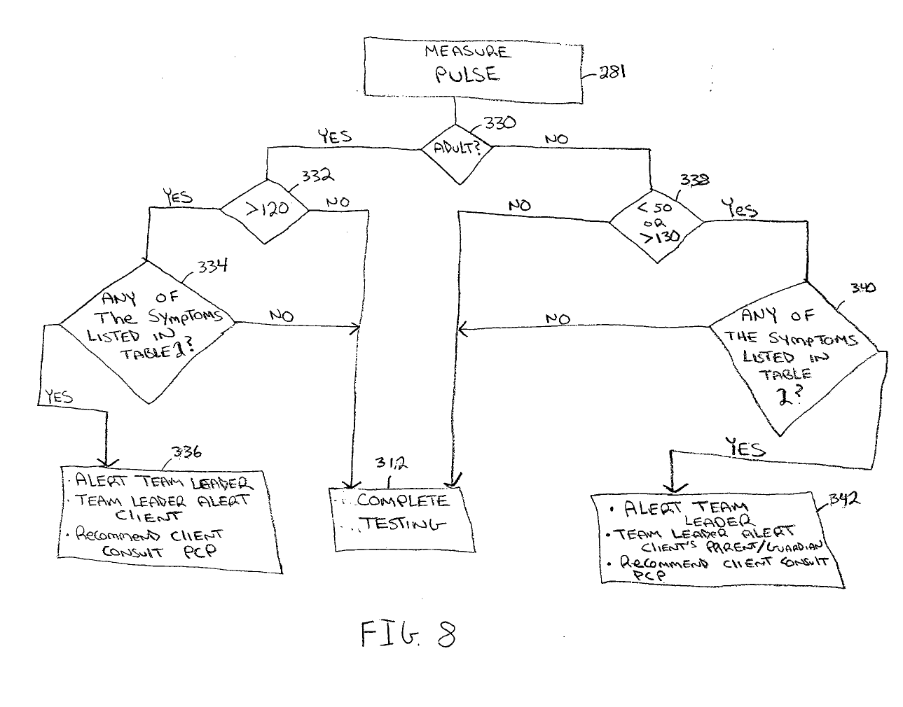

In addition to recognizing emergency situations resulting from abnormal ECG testing, a clinician and/or Team Leader can identify emergency situations arising from abnormal pulse and/or blood pressure readings. For example, FIG. 8 provides an embodiment of steps a clinician can follow to identify an abnormal pulse rate. As shown, following testing (Step 281), a clinician can determine if the client is an adult (i.e., at least 18 years of age) (Step 330). If yes, the clinician can determine if the pulse rate is greater than 120 beats/minute (Step 332). If no, the clinician can record the data and complete testing (Step 312). If yes, the clinician can determine if client is exhibiting any of the symptoms listed in Table 2 above (Step 334). If no, the clinician can record the data and complete testing. If yes, the clinician can inform the Team Leader and the Team Leader can inform the client of the test data and recommend the client seek medical attention. If the client is not an adult (see Step 330), the clinician can determine if the pulse rate is either less than 50 beats/minute or greater than 130 beats/minute. If no, the clinician can record the results and complete testing (Step 312). If yes, the clinician can determine if client is exhibiting any of the symptoms listed in Table 2 above (Step 340). If no, the clinician can record results and complete testing (Step 312). If yes, the clinician can inform Team Leader and Team Leader can inform client and recommend client seek medical attention.

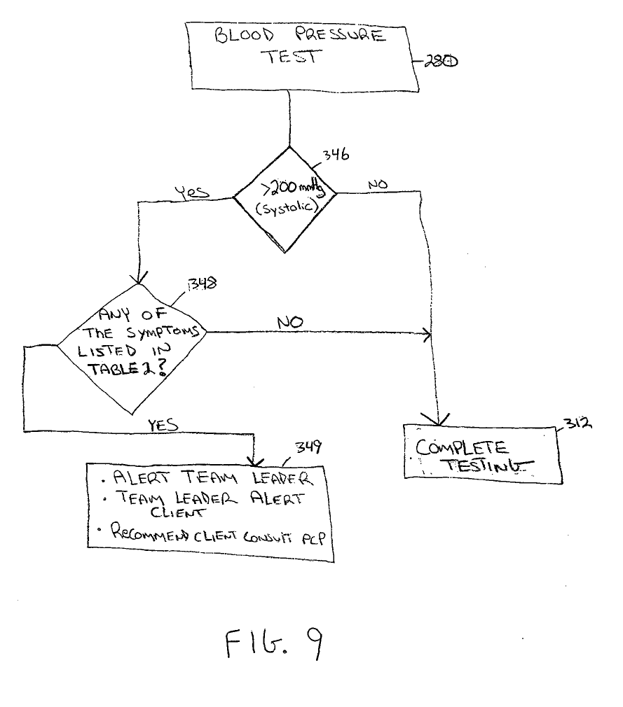

Similarly, FIG. 9 provides a representation of an embodiment for identifying and classifying an abnormal blood pressure result. Following blood pressure testing (Step 280), a clinician can determine if blood pressure result is greater than 200 mm Hg. If no, a clinician can record the results and complete testing (Step 312). If yes, the clinician can determine if the client is exhibiting any of the symptoms listed in Table 2 above. If no, the clinician can record the result and complete testing (Step 312). If yes, the clinician can alert Team Leader who can alert client and recommend client seek medical attention (Step 349).

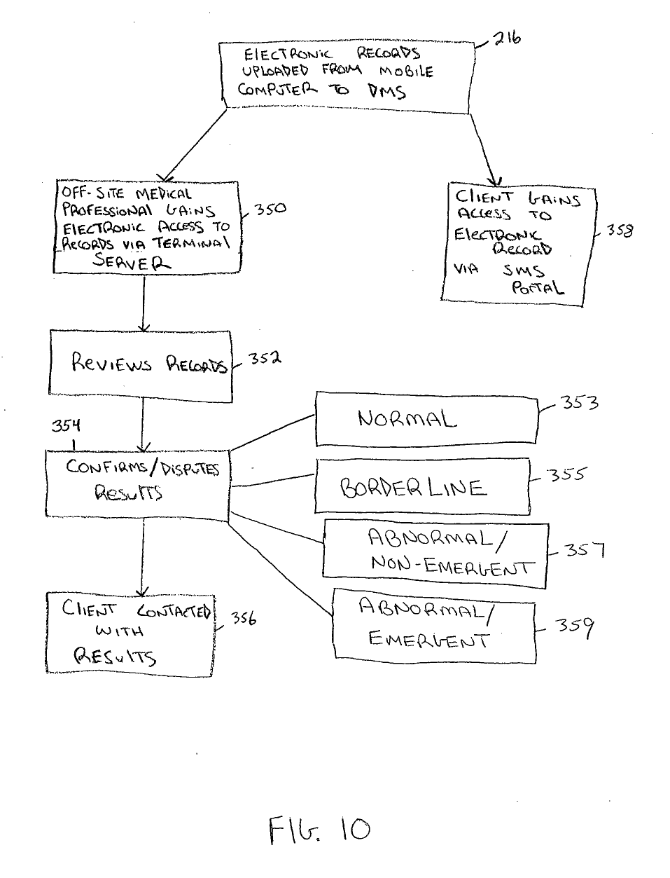

Following testing and identification of emergency situations, all electronic records 114, as illustrated in FIG. 10, can be uploaded from the mobile computer unit 32 to the DMS 22 (Step 216). Further, all client data can be removed (i.e., erased/deleted) from the mobile computing units 32, 34, 36, 38, 40 in order to maintain security of client data. Over the course of the next several days, the off-site cardiologist can review (i.e., over-read) all electronic records (both normal and abnormal) by accessing the DMS 22 via a terminal server 26 (Step 350). As such, the system 10 includes an verification step wherein the off-site medical professional can review all records (Step 354) and confirm or dispute the initial results (Step 354). In disputing or confirming, the off-site cardiologist can classify the client results as follows: Normal (Step 353); Borderline (Step 355); Abnormal/Non-Emergent (Step 357); or Abnormal/Emergent (Step 359). Following such review and classification, the DMS 22 can produce a report and a summary letter to be sent to the client. Furthermore, the system 10 can be adapted so as to allow a client access to the client's electronic record via the SMS portal 24 (Step 358). As such, the client can provide their electronic record to the client's personal care physician for further analysis and medical attention.

The invention being thus disclosed and illustrative embodiments depicted herein, further variations and modifications of the invention will occur to those skilled in the art. All such variations and modifications are considered to be within the scope of the invention, as defined by the claims appended hereto and equivalents thereof.

What is claimed is:

1. A method of screening a population for a medical condition, comprising:
(A) establishing a testing site at a location remote from a healthcare facility, the testing operation including at least one healthcare clinician and at least one mobile computing unit;
(B) obtaining or creating an electronic medical record for a client from among the population and storing that electronic medical record on the at least one mobile computing unit;
(C) testing of the client for the condition by the healthcare clinician using a digital medical device connected to the mobile computing device;
(D) adding data from the testing to the client's electronic medical record;
(E) uploading the client's electronic medical record from the mobile computing device to a Data Management Server associated with the healthcare facility; and
(F) providing authorized end-users with access to the client's electronic medical record.

2. The method of claim 1, wherein steps (A) through (F) are repeated to test the entire population.

3. The method of claim 1, wherein a plurality of healthcare clinicians, each having a testing computing device in communication with the mobile computing device, the testing device coupled to a digital medical device.

4. The method of claim 3, wherein each healthcare clinician has two digital medical devices so that one client from the population can be prepared for testing while another client from the population is being tested.

5. The method of claim 3, wherein the testing center includes an storage device in communication with the mobile computing device.

6. The method of claim 1, wherein the digital medical device is an electrocardiogram device.

7. The method of claim 6, wherein the medical condition is one indicative of a heightened risk of sudden cardiac death.

8. The method of claim 1, wherein the electronic medical record, including testing data, is forwarded electronically to an on-call medical professional at a remote location at least for clients whose testing indicates a likelihood that the medical condition is present.

9. The method of claim 1, wherein the testing location is a school.

10. The method of claim 9, wherein the client population are students.

11. A method for testing a client population for a medical condition, comprising:
providing an SMS portal, the SMS portal allowing a client to establish an electronic client medical record by providing various types of information via an internet connection, the electronic medical record stored in a data management server;
down-loading the electronic medical records to a mobile computer unit, the mobile computer unit capable of being moved to an off-site testing facility;
coupling a digital medical device to the mobile computer unit, the digital medical device capable of performing a client test and incorporating test date to the electronic client record to produce a updated electronic record;
up-loading the updated electronic client records from the mobile computer unit to the data management server; and
providing an off-site medical professional with electronic access to the electronic client record via a terminal server, the terminal server being coupled to the data management server.

12. The method of claim 11 further comprising:
providing the client with access to the client's electronic medical record via the internet connection.

13. The method of claim 11, wherein the digital medical device is a digital ECG device.

14. The method of claim 11, wherein the medical condition is a cardiac condition.

15. The method of claim 11, wherein the client population are student athletes.

16. The method of claim 15, wherein the testing location is a school.

17. A system for screening a population of clients for a medical condition, comprising:
a data management server located at a healthcare facility, the data management server in communication with an SMS portal, the SMS portal assessable to a client via an internet connection, the data management server capable of creating an electronic client record containing various information provided by the client via the SMS portal;
an at least one mobile computer unit capable of moving from the healthcare facility to an off-site testing location, the mobile computer unit capable of receiving electronic records downloaded from the data management server, the mobile computer unit coupled to a digital medical device capable of performing a client test and directly incorporating a test result into the client's electronic record, the mobile computer unit further capable of uploading the electronic medical record to the data management server; and
a terminal server capable of allowing an off-site medical professional access to the electronic medical record.

18. The system of claim 17 further comprising a plurality of testing computer units in communication with the mobile computer unit.

19. The system of claim 17, wherein the medical condition is a cardiac condition.

20. The system of claim 19, wherein the digital medical device is an ECG device.
